(19) United States
(12) Patent Application Publication
(10) Pub. No.: US 2004/0141951 A1
(43) Pub. Date: Jul. 22, 2004

(54) Cytokine inhibition of eosinophils

(76) Inventors: Marc Elliot Rothenberg, Cincinnati, OH (US); Patricia Chandhok Fulkerson, Cincinnati, OH (US)

Correspondence Address: WOOD, HERRON & EVANS, LLP, 2700 CAREW TOWER, 441 VINE STREET, CINCINNATI, OH, 45202, US

(21) Appl. No.: 10/752,659

(22) Filed: Jan. 7, 2004

Related U.S. Application Data
(60) Provisional application No. 60/438,412, filed on Jan. 7, 2003.

(51) Int. Cl. A61K 38/19

(52) U.S. Cl. 424/85.1

(57) ABSTRACT

A composition and method to alter eosinophil function and recruitment. An allergen-induced chemokine with inhibitory activity on eosinophils, monokine induced by interferon γ (MIG) and/or an IFN-γ-inducible protein of 10 kDa (IP-10), is administered in a pharmaceutically acceptable dose and formulation. The composition is used for prophylaxis and therapy of diseases in which eosinophilia occurs and may be administered, for example, to allergic patients and asthmatic patients.

### RELATED APPLICATION

This application claims priority to U.S. Provisional Patent Application Serial No. 60/438,412 filed Jan. 7, 2003, now pending and expressly incorporated by reference herein in its entirety.

The U.S. Government has a paid-up license in this invention and the right in limited circumstances to require the patent owner to license others on reasonable terms as provided for by the terms of Grant Nos. RO1 AI42242-04XX and AI45898 awarded by the National Institutes of Health.

### FIELD OF THE INVENTION

The invention is directed to compositions and methods of chemoattractant-induced alteration of eosinophil function and distribution.

### BACKGROUND

Eosinophils are one type of granulocytic leukocyte (white blood cell) or granulocyte that normally appears in the peripheral blood at a concentration of about 1-3% of total leukocytes. Their presence in tissues is normally primarily restricted to the gastrointestinal mucosa. In various disease states, eosinophils are increased in the peripheral blood and/or tissues, a condition termed eosinophilia and described by Rothenberg in Eosinophilia, N. Engl. J. Med. 338, 1592-1600 (1998).

Eosinophil accumulation in the peripheral blood and tissues is a hallmark feature of several diseases. These diseases include allergic disorders such as allergic rhinitis, asthma, and eczema; parasitic infections; certain types of malignancies; chronic inflammatory disorders such as inflammatory bowel disease; and specific syndromes such as eosinophilic gastroenteritis, eosinophilic colitis, eosinophilic cellulitis, eosinophilic esophagitis, eosinophilic fascitis; and systemic diseases such as Churg Strauss syndrome, eosinophilic pneumonia, and the idiopathic hypereosinophilic syndrome. Eosinophil accumulation in tissues may cause potent pro-inflammatory effects or tissue remodeling.

Numerous mediators have been identified as eosinophil chemoattractants. These include diverse molecules such as lipid mediators (platelet activating factor (PAF), leukotrienes) and recently chemokines, such as the eotaxin subfamily of chemokines. Chemokines are small secreted proteins produced by tissue cells and leukocytes that regulate leukocyte homing during homeostatic and inflammatory states. Two main subfamilies (CXC and CC chemokines) are distinguished depending upon the arrangement of the first two cysteines, which are separated by one amino acid (CXC) or are adjacent (CC).

The finding that eosinophils normally account for only a small percentage of circulating or tissue dwelling cells, and that their numbers markedly and selectively increase under specific disease states, indicates the existence of molecular mechanisms that regulate the selective generation and accumulation of these leukocytes. A composition to regulate eosinophil function would therefore be desirable, in view of the wide variety of eosinophil-mediated conditions. For example, pediatric asthma is an eosinophil-mediated condition whose incidence is on the rise and is now the chief diagnosis responsible for pediatric hospital admissions. Alleviation of asthma, along with the spectrum of other eosinophil-mediated conditions, by altering eosinophil function would be of benefit.

### SUMMARY OF THE INVENTION

One embodiment of the invention is directed to a method of inhibiting eosinophil function. A pharmaceutical composition containing an isolated cytokine with eosinophil function-inhibitory activity is administered to a patient in a pharmaceutically effective amount to inhibit eosinophil function. The composition may inhibit receptor expression, receptor internalization, signal transduction, transmigration, desensitization, degranulation, and/or mediator release. The cytokine may be monokine induced by interferon γ (MIG), and/or IFN-γ-inducible protein of 10 kDa (IP-10).

Another embodiment of the invention is a method of reducing allergen-induced eosinophilia, for example, in an airway, the lungs, the trachea, the bronchoalveolar lavage fluid, or the blood. Eosinophilia may also be reduced in a body part affected by an allergy, such as eyes, skin, and gut.

Another embodiment of the invention is a treatment method by administering a pharmaceutical composition containing an eosinophil-inhibitory cytokine in an amount sufficient to inhibit an eosinophil response to a chemoattractant. The cytokine may be MIG and/or IP-10, administered at a dose of about 10 μg/kg to about 10 mg/kg, and the chemoattractant may be eotaxins-1, -2, or -3, MCP-2, -3, -4, or -5, RANTES, and/or MIP-1a. The dose may be systemically administered, for example, intravenously or orally.

Another embodiment of the invention is a palliative method whereby a pharmaceutical composition containing an isolated eosinophil-inhibitory cytokine is administered in an amount to alleviate inflammation in the airway of a patient that is likely or be or that has been exposed to an allergen. The patient may exhibit symptoms of rhinitis, asthma, and/or eczema.

Another embodiment of the invention is a method of inhibiting pulmonary eosinophil recruitment by administering MIG and/or IP-10 in a pharmaceutical composition in an amount to inhibit pulmonary eosinophil recruitment. The patient administered the composition may be asthmatic and/or allergic.

Another embodiment of the invention is a treatment method for an allergic patient by administering a pharmaceutical composition containing at least one cytokine capable of negatively regulating a pulmonary inflammatory cell.

Another embodiment of the invention is a treatment method for an individual with eosinophilia. Either MIG and/or IP-10 is administered at a dose of up to about 10 mg/kg to alter eosinophil migration, tissue recruitment, receptor binding, signal transduction, degranulation, and/or mediator release.

Another embodiment of the invention is a method for alleviating asthma in a patient. MIG is administered in a pharmaceutical composition, thereby inhibiting an interleukin (IL)-13-associated asthmatic response in the patient.

Another embodiment of the invention is a pharmaceutical composition containing MIG and/or IP-10 in a pharmaceutically acceptable formulation and an amount sufficient to alter eosinophil activity in the presence of an allergen. The amount is such that a dose from about 10 μg/kg to about 10 mg/kg can be administered.

Another embodiment of the invention is a pharmaceutical composition containing a cytokine which inhibits at least one eosinophil function in response to an eosinophil-induced stimulus. The cytokine may be MIG and/or IP-10. The stimulus may be an allergen, an allergic reaction, an infection, and/or a chemokine such as eotaxin, IL-13, and/or platelet activating factor. Alternatively, the stimulus may be idiopathic.

Another embodiment of the invention is a pharmaceutical composition containing an isolated Th1-associated chemokine in a pharmaceutically acceptable formulation and in an amount sufficient to inhibit eosinophil activity in the presence of an allergen.

Another embodiment of the invention is a pharmaceutical composition containing a recombinant MIG and/or recombinant IP-10 cytokine in a pharmaceutically acceptable formulation and dose sufficient to inhibit an eosinophil function.

These and other advantages will be apparent in light of the following figures and detailed description.

### BRIEF DESCRIPTION OF THE DRAWINGS

This application contains at least one drawing executed in color. A Petition under 37 C.F.R. §1.84 requesting acceptance of the color drawings is filed separately on even date herewith.

FIG. 1 demonstrates allergen induction of the cytokines monokine-induced interferon γ (MIG) and IFN-γ-inducible protein of 10 kDa (IP-10). FIGS. 1A and 1B are graphs from microarray hybridization analysis showing induction of monokine-induced interferon γ (MIG) (FIG. 1A) and IP-10 (FIG. 1B) in mice with experimental asthma from control and challenged lung with the ovalbumin allergen. FIG. 1C is a Northern blot of lung ribonucleic acid (RNA) from control and challenged lung at various time points after allergen exposure. FIG. 1D is a Northern blot of lung RNA from control and challenged lung with Aspergillus as the allergen. FIG. 1E is a Northern blot showing the effect of the transcription factor STAT-6 on MIG induction following Aspergillus challenge in wild type and knockout mice. FIG. 1F(A-D) shows the expression pattern of MIG mRNA in ovalbumin-challenged lung by in-situ hybridization.

FIG. 2 shows comparative receptor expression data. FIG. 2 (A-D) shows data from flow cytometry analysis. FIG. 2E is a representative chemotaxis assay showing the effect of MIG on eosinophil migration in vitro.

FIG. 3 shows the inhibitory effect of MIG-pretreatment on eosinophil chemotactic response to eotaxin-2 in vitro.

FIG. 4 shows the effect of MIG-pretreatment on eosinophil migration to lung in vivo. FIG. 4A shows the effect of MIG-pretreatment on eosinophil response to eotaxin-2. FIG. 4B shows the effect of increasing doses of MIG. FIG. 4C shows the effect of eotaxin-1 pretreatment on eosinophil response to eotaxin-2. FIGS. 4D(1-2) show lung tissue with eosinophils detected by anti-MBP immunohistochemistry. FIG. 4E shows the effect of MIG on eotaxin-induced eosinophil mobilization to the blood.

FIG. 5 shows the effect of MIG on eosinophil recruitment to the lung in ovalbumin-induced experimental asthma. FIG. 5A shows the effect of MIG pretreatment on eosinophil recruitment in response to the allergen ovalbumin. FIG. 5B shows the effect of neutralizing MIG prior to ovalbumin challenge using control and anti-MIG antibodies.

FIG. 6 shows the effect on eosinophils of MIG in eosinophils in vitro. FIGS. 6A(1-2) show the specific binding of MIG to eosinophils. FIGS. 6B(1-2) show the lack of internalization of CCR3 by MIG. FIGS. 6C(1-2) show Western blots of eosinophils exposed to eotaxin-2 in the presence and absence of MIG pretreatment, with levels of phosphorylated and total Erk1 and Erk2 shown. FIG. 6D shows the effect of MIG on superoxide production.

FIG. 7 shows the effect of MIG on chemotaxis of eosinophils toward non-CCR3 ligands.

FIG. 8 shows the effect of MIG on leukocyte recruitment to the lung induced by the cytokine IL-13.

### DETAILED DESCRIPTION

Chemokines which specifically alter eosinophil function, and methods for their pharmaceutical use, are disclosed. They include monokine induced by interferon-γ (MIG), and an IFN-γ-inducible protein of 10 kDa (IP-10). Their role in therapy for eosinophil-associated diseases and mechanisms of action are also disclosed. As will be appreciated by one skilled in the art, the term cytokine will be used herein to encompass a chemokine.

Chemokines induce signals via seven transmembrane-domain receptors coupled to G proteins, which also form two main subfamilies for CXC and CC chemokines, designated CXCR⁶ and CCR, respectively. Eotaxin (CCL11; now designated eotaxin-1), a CC chemokine with selective activity on eosinophils, has a dominant role in regulating eosinophil baseline homing, and a contributory role in regulating eosinophil tissue recruitment during allergen-induced inflammatory responses. Additional chemokines have been identified in the genome which encode for CC chemokines with eosinophil-selective chemoattractant activity, and have been designated eotaxin-2 (in humans and mice) and eotaxin-3 (in humans only).

The specific activity of eotaxins-1, -2, and -3 is mediated by the selective expression of the eotaxin receptor, CCR3, on eosinophils. CCR3 is a promiscuous receptor; it interacts with multiple ligands including macrophage chemoattractant proteins (MCP)-2, -3, and 4, RANTES (regulated upon activation normal T-cell expressed and secreted), and HCC-2 (MIP-5, leukotactin); however, the only ligands that signal exclusively through this receptor are eotaxins-1, -2, and -3, accounting for the cellular selectivity of the eotaxins. CCR3 appears to function as the predominant eosinophil chemokine receptor because CCR3 ligands are generally more potent eosinophil chemoattractants. Furthermore, an inhibitory monoclonal antibody specific for CCR3 blocks the activity of RANTES, a chemokine that could signal through CCR1 or CCR3 in eosinophils. Other cells involved in allergic responses, Th2 cells, basophils, mast cells, and possibly respiratory epithelial cells also express CCR3; however, the significance of CCR3 expression on these cells has been less clearly demonstrated than on eosinophils.

During induction of eosinophil-associated allergic airway inflammation, leukocyte tissue recruitment is orchestrated by the coordinated induction of chemokines. Focusing on eosinophils, a paradigm has emerged implicating Th2 cytokines in the induction of eosinophil active chemokines. For example, IL-4 and IL-13 are potent inducers of eotaxins and MCPs in vitro. When Th2 cytokines are over-expressed or administered to the lung, there is marked induction of eotaxins, as well as strong eosinophil lung recruitment.

In contrast, Th1 cytokines, such as interferon-γ (IFN-1) induce a different set of chemokines (e.g. an IFN-γ-inducible protein of 10 kDa termed IP-10 or CXCL10; monokine induced by interferon termed MIG or CXCL9; and IFN-inducible T cell a chemoattractant termed I-TAC or CXCL11). These chemokines are unique in that they selectively signal through CXCR3, a receptor expressed on activated T cells (preferentially of the Th1 phenotype), on NK cells, and a significant fraction of circulating CD4 and CD8 T cells. This dichotomy may be even further complex in view of a recent publication indicating that human CXCR3 ligands are human CCR3 antagonists, inhibiting the action of CCR3 ligands on CCR3⁺ cells in vitro (Loetscher et al., J. Biol. Chem. 276:2986 (2001)).

Mice overexpressing murine IL-4 under the regulation of the Clara cell 10 promoter (a kind gift of Dr. Jeffrey Whitsett) were used to examine induction of MIG (Rankin et al., Proc. Natl. Acad. Sci. U.S.A. 93:7821 (1996)). All mice were maintained under specific pathogen free conditions and according to institutional guidelines. IL-5 transgenic mice were used as a source of blood and spleen eosinophils.

Asthma was experimentally induced in mice using both ovalbumin (OVA)-induced and Aspergillus fumigatus asthma models. These models are described in Mishra et al., J. Clin. Invest. 107:83 (2001), which is expressly incorporated by reference herein in its entirety. Briefly, for OVA-induced asthma, mice were injected intraperitonally (i.p.) with both OVA and aluminum hydroxide (alum) (1 mg) adjuvant on days 0 and 14, followed by an intranasal OVA or saline challenge on day 24. For Aspergillus-induced asthma, mice received repeated intranasal administrations of Aspergillus fumigatus over the course of three weeks.

Eosinophilia was induced by administration of either eotaxin-2 or IL-13, using procedures described in Rothenberg et al., Molec. Med. 2:334 (1996), which is expressly incorporated by reference herein in its entirety. Mice received 3 μg of recombinant eotaxin-2 (a kind gift of Peprotech, Rocky Hill N.J.), or 4 μg and 10 μg of IL-13, directly into the lung via intratracheal delivery. Briefly, mice were anesthetized with ketamine (5 mg/100 μl) then were positioned upright, after which 20 μl of recombinant eotaxin-2, IL-13, or saline (control) was delivered into the trachea with a pipette (Pipetman®, Gilson, Middleton Wis.).

For delivery of MIG, 200 μl (1 μg) of the recombinant chemokine (Peprotech) was injected into the lateral tail vein 30 minutes prior to the intratracheal or intranasal administration of eotaxin-2 and/or intranasal challenge administration of OVA.

MIG neutralization in OVA-sensitized mice was induced with an intraperitoneal injection of 500 μl (500 μg) of anti-murine MIG (a kind gift of Joshua M. Farber) twenty-four hours prior to a single challenge of OVA or saline. Control groups were injected with an isotope-matched control antibody.

Bronchoalveolar lavage fluid (BALF) and/or lung tissue from allergen-challenged mice was harvested 18 hours after challenge. Mice were euthanized by CO₂ inhalation, a midline neck incision was made, and the trachea was cannulated. The lungs were lavaged twice with 1.0 ml phosphate buffered saline (PBS) containing 1% fetal calf serum (FCS) and 0.5 mM ethylenediaminetetraacetic acid (EDTA). The BALF recovered was centrifuged (400×g for 5 minutes at 4° C.) and resuspended in 200 μl PBS containing 1% FCS and 0.5 mM EDTA. Total cell numbers were counted with a hemocytometer. Cytospin preparations were stained with Giemsa-Diff-Quick (Dade Diagnostics of P.R., Inc., Aguada PR) and differential cell counts were determined. Ribonucleic acid (RNA) from lung was extracted using the Trizol reagent as per the manufacturer's instructions. Following Trizol purification, RNA was repurified with phenol-chloroform extraction and ethanol precipitation.

Microarray hybridization was performed by the Affymetrix Gene Chip Core facility at Children's Hospital Medical Center (Cincinnati Ohio). Briefly, RNA quality was first assessed using the Agilent bioanalyzer (Agilent Technologies, Palo Alto Calif.). Only mRNA having a ratio of 28S/18S between 1.3 and 2 were subsequently used. RNA was converted to cDNA with Superscript choice for cDNA synthesis (Invitrogen, Carlsbad Calif.) and subsequently converted to biotinylated cRNA with Enzo High Yield RNA Transcript labeling kit (Enzo Diagnostics, Farmingdale N.Y.). After hybridization to the murine U74Av2 GeneChip (Affymetrix, Santa Clara Calif.), the chips were automatically washed and stained with streptavidin-phycoerythrin using a fluidics system. The chips were scanned with a Hewlett Packard GeneArray Scanner. The analysis was performed with one mouse per chip (n=3 for each allergen challenge condition, and n=2 for each saline challenge condition).

Levels of gene transcripts were determined from data image files, using algorithms in the Microarray Analysis Suite Version 4 software (Affymetrix). Levels from chip to chip were compared by global scaling; thus, each chip was normalized to an arbitrary value (1500). Each gene is typically represented by a probe set of 16 to 20 probe pairs. Each probe pair consists of a perfect match oligonucleotide and a mismatch oligonucleotide that contains a one base mismatch at a central position. Two measures of gene expression were used, absolute call and average difference. Absolute call is a qualitative measure in which each gene is assigned a call of present, marginal or absent, based on the hybridization of the RNA to the probe set. Average difference is a quantitative measure of the level of gene expression, calculated by taking the difference between mismatch and perfect match of every probe pair and averaging the differences over the entire probe set. Differences between saline and OVA-treated mice were also determined using the GeneSpring software (Silicon Genetics, Redwood City Calif.). Data for each allergen challenge time point were normalized to the average of the saline-treated mice. Gene lists were created with results having p<0.05 and >2-fold change.

Lung tissue samples were fixed with 4% paraformaldehyde in phosphate buffer (pH 7.4), embedded in paraffin, cut into 5 μm sections, and fixed to positive charge slides. For analysis of mucus production, tissue was stained with Periodic Acid Schiff (Poly Scientific R&D Corp.) according to the manufacturer's recommendations. Specifically, a 1.0×1.0 cm grid ocular was used to quantify the percent of epithelial cells that were producing mucus. Five hundred linear gradations (representing 6.25 mm of epithelium) were randomly counted, and the results were expressed as a ratio of mucus producing cells:total pulmonary epithelial cells. For eosinophil staining in tissue, an antiserum against murine major basic protein (anti-MBP) was applied, as described in Matthews et al., Proc. Natl. Acad. Sci. U.S.A. 95:6273 (1998), which is expressly incorporated by reference herein in its entirety. In brief, endogenous peroxidase was quenched with 0.3% hydrogen peroxide in methanol, followed by non-specific protein blocking with normal goat serum. Tissue sections were then incubated with rabbit anti-murine MBP antibody (1:2500, a kind gift from J. Lee, Mayo Clinic, Scottsdale Ariz.) overnight at 4° C., followed by biotinylated goat anti-rabbit IgG secondary antibody (1:200 dilution) and avidin-peroxidase complex (Vector Laboratories) for thirty minutes each. These slides were further treated with nickel diaminobenzidine-cobalt chloride solution to form a black precipitate, and counter-stained with nuclear fast red. Immunoreactive cells were quantitated by morphometric analysis (Metamorph Imaging System, Universal Imaging Corporation, West Chester Pa.) as described in Mishra et al., J. Clin. Invest. 107:83 (2001). The lung sections were taken from the same position in each set of mice and at least 4-5 random sections/mouse were analyzed. Using digital image capture, tissue regions associated with medium sized bronchioles or blood vessels were quantified for the total MBP⁺ cell units relative to the total tissue area. Calculated eosinophil levels were expressed as cells/mm².

Chemotactic responses were determined by transmigration through respiratory epithelial cells as previously described in Zimmermann et al., J. Immunol. 164:1055 (2000), which is expressly incorporated by reference herein in its entirety. In brief, A549 cells (American Type Tissue Culture Collection, Rockville Md.) were grown as monolayers in tissue-culture flasks in Dulbecco's Modified Eagles Medium (DMEM) (Gibco BRL) supplemented with 10% FCS, penicillin, and streptomycin. Cell monolayers were trypsinized, centrifuged, and resuspended in fresh medium prior to culture on permeable filters (polycarbonate filters with 3 μm pores) in Transwell tissue-culture plates (Corning Costar Corp., Cambridge Mass.). Cells (1.5×10⁵) in a volume of 100 μl were grown to confluence on the upper surface of the filters for two days, and treated with 10 ng/ml TNFα for 18 hours. Leukocytes (1.5×10⁶) in Hank's buffered salt solution (HBSS) and 0.5% bovine serum albumin (BSA, low endotoxin, Sigma) were placed in the upper chamber and the chemokine (in HBSS and 0.5% BSA) was placed in the lower chamber. Eosinophils were obtained using splenocytes from IL-5 transgenic mice. Transmigration was allowed to proceed for 1.5 hours. Cells in the lower chamber were counted in a hemocytometer, cytocentrifuged, stained with Giemsa-Diff-quick (Dade Diagnostics of P.R., Inc., Aguada PR), and the differential white cell analysis was determined microscopically.

For flow cytometry analysis, splenocytes (5×10⁵) were washed with FACS-buffer (2% BSA, 0.1% Na-azide in PBS) and incubated for 20 minutes at 4° C. with one of the following: 150 ng (1.5 μg/ml) phycoerythrin-conjugated anti-murine CCR-3 antibody (R&D Systems, Minneapolis Minn.), 300 ng (3 μg/ml) anti-murine CXCR3 (a kind gift of Merck Research Laboratories), 1 μg (10 μg/ml) FITC-conjugated anti-murine CD4 (BD Biosciences Pharmingen, San Diego Calif.), or isotope-matched control IgG. After two washes in FACS-buffer, cells stained for CXCR3 were incubated in the dark with 0.3 μg FITC-conjugated isotope specific secondary antibody (Pharmingen) for 20 minutes at 4° C. After two washes, labeled cells were subjected to flow cytometry on a FACScan flow cytometer (Becton Dickinson) and analyzed using the CELLQuest software (Becton Dickinson). Internalization of surface CCR3 was assayed as described in Zimmermann et al., J. Biol. Chem. 274:12611 (1999), which is expressly incorporated by reference herein in its entirety. Briefly, cells were incubated for 15 minutes at either 4° C. or 37° C., with either 0 or 100 ng/ml murine eotaxin-2, or with 1-1000 ng/ml murine MIG. Following chemokine exposure, cells were immediately placed on ice and washed with at least twice the volume of cold FACS buffer.

For MIG binding, cells (5×10⁵) were incubated for 15 minutes at 4° C. with 100 nM to 1000 nM murine MIG. Following chemokine exposure, cells were washed, fixed with 2% paraformaldehyde, washed, and then incubated with 500 ng (5 μg/ml) anti-murine MIG (R&D Systems) at 4° C. for 15 minutes. Cells were stained with phycoerythrin-conjugated secondary antibody. For eotaxin binding, cells (1×10⁶) were exposed to 0 μM to 10 μM of MIG or eotaxin-2 for five minutes at 4° C. prior to incubation with 20 nM biotinylated eotaxin (Sigma) or JE (R&D Systems). Chemokine binding was detected with FITC-conjugated avidin (R&D Systems).

Eosinophils were purified from splenocytes from IL-5 transgenic mice for signal transduction studies following depletion of T and B cells using Dynabeads Mouse pan B (B220) and pan T (Thy 1.2) per the manufacturer's instructions. Purified (85%) eosinophils (1×10⁶) were incubated in RPMI Medium 1640 (Invitrogen Corporation, Carlsbad Calif.) for fifteen minutes at 37° C. prior to stimulation with 10 nM eotaxin-2 and/or 50 nM to 500 nM MIG for two or ten minutes at 37° C. Reactions were stopped with cold PBS with 2 mM sodium orthovanadate (Sigma). Cells were lysed in 25 μl of lysis buffer (5 mM EDTA, 50 mM NaCl, 50 mM NaF, 10 mM Tris-HCl pH 7.6, 1% Triton-X, 0.1% BSA). An equal volume of sample buffer was added to each lysate prior to boiling for five minutes. Samples were separated on a NuPAGE 4-12% Bis-Tris SDS gel (Invitrogen). The proteins were transferred by electroblotting onto nitrocellulose membranes (Invitrogen). The blots were probed with antibodies specific for (1:1000) phospho-p44/42 Map Kinase (Cell Signaling Technologies, Beverly Mass.). Membranes were stripped by incubation at 50° C. for thirty minutes in stripping buffer (100 mM 2-mercaptoethanol, 2% SDS, 62.5 mM Tris-HCl pH 6.7), and then reprobed with antibodies to (1:1000) p44/42 (Cell Signaling Technologies). Proteins were visualized using the ECL system (Amersham Pharmacia Biotech, Piscataway N.J.) after incubating membranes with (1:1500) anti-rabbit IgG HRP (Cell Signaling Technologies).

Data were expressed as mean ± standard deviation except where noted. Statistical significance comparing different sets of mice was determined by the Student's t-test.

Identification of CXCR3 Ligands in Experimental Asthma

Genes differentially expressed in a well established model of asthma were identified. To induce asthma, mice were intraperitonally sensitized with the allergen OVA in the presence of the adjuvant alum on two separate occasions separated by 14 days. Subsequently, replicate mice were challenged with intranasal OVA or control saline on two occasions separated by 3 days. Three and/or eighteen hours after each allergen challenge, lung RNA was subjected to microarray analysis utilizing the Affymetrix chip U74Av2 that contained oligonucleotide probe sets representing 12,423 genetic elements, the largest collection of characterized mouse genes commercially available.

Comparison of allergen-challenged mice to saline-challenged mice revealed a >2-fold change in 2-6% of the genes, at the various time points (data not shown). Of these allergen-induced genes, the genes encoding chemokines represented a large subset. For example, the microarray chip contained oligonucleotides that represented 29 chemokine genes; 10 of these were allergen-induced, compared with saline challenged control mice. Several of the induced chemokine genes were not previously associated with allergic lung responses. For example, there was strong induction of the IFN-γ inducible chemokines MIG and IP-10.

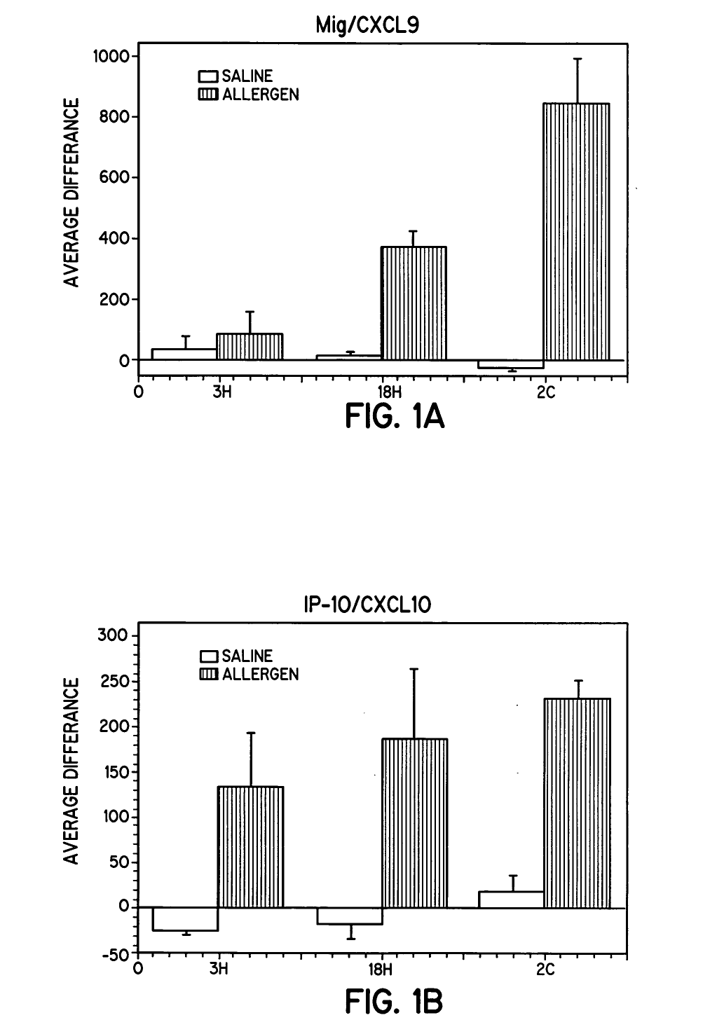

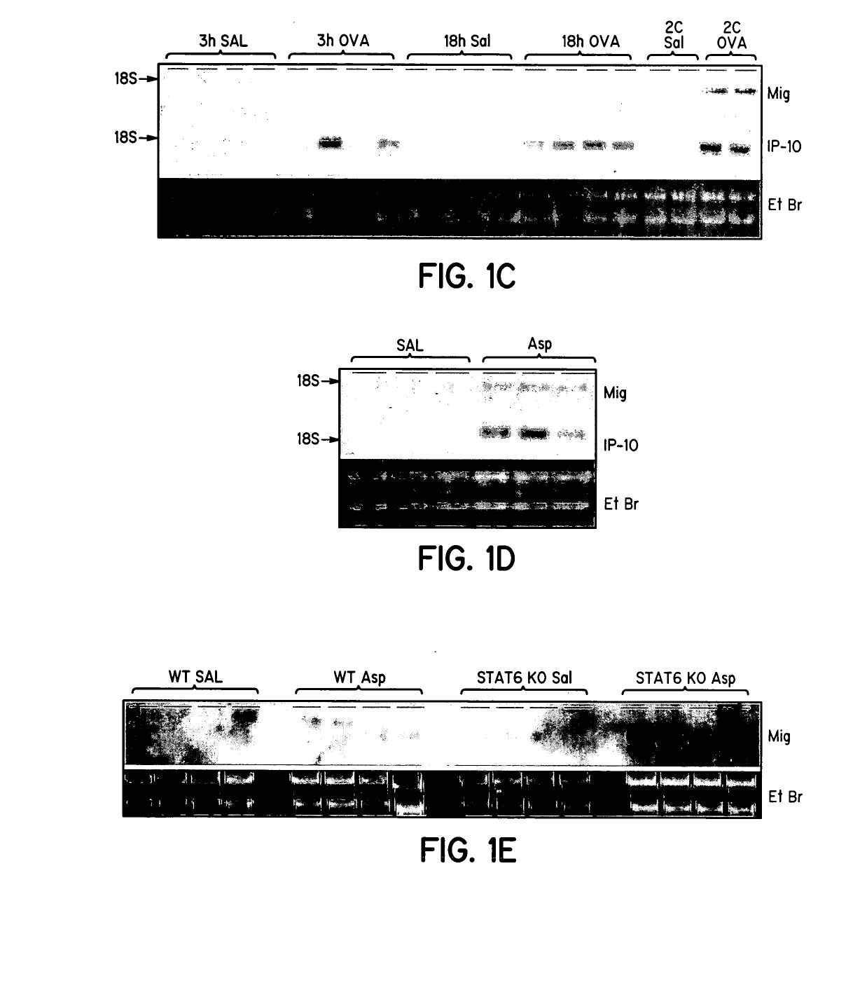

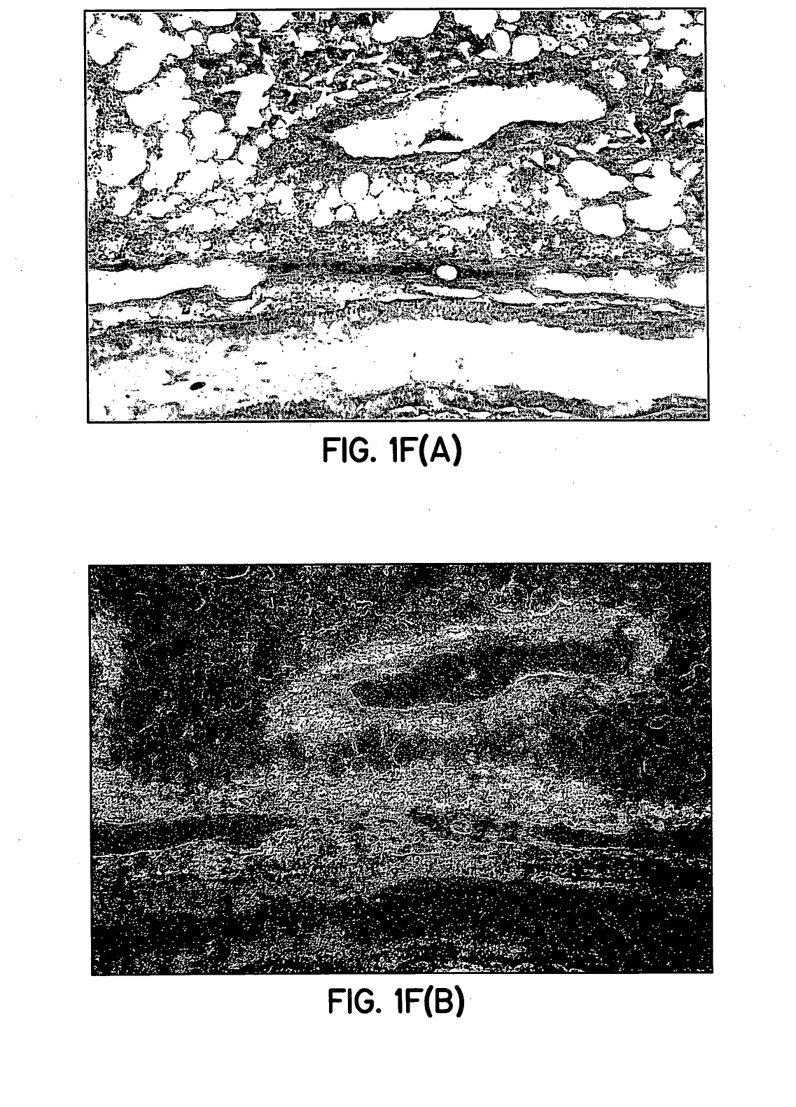

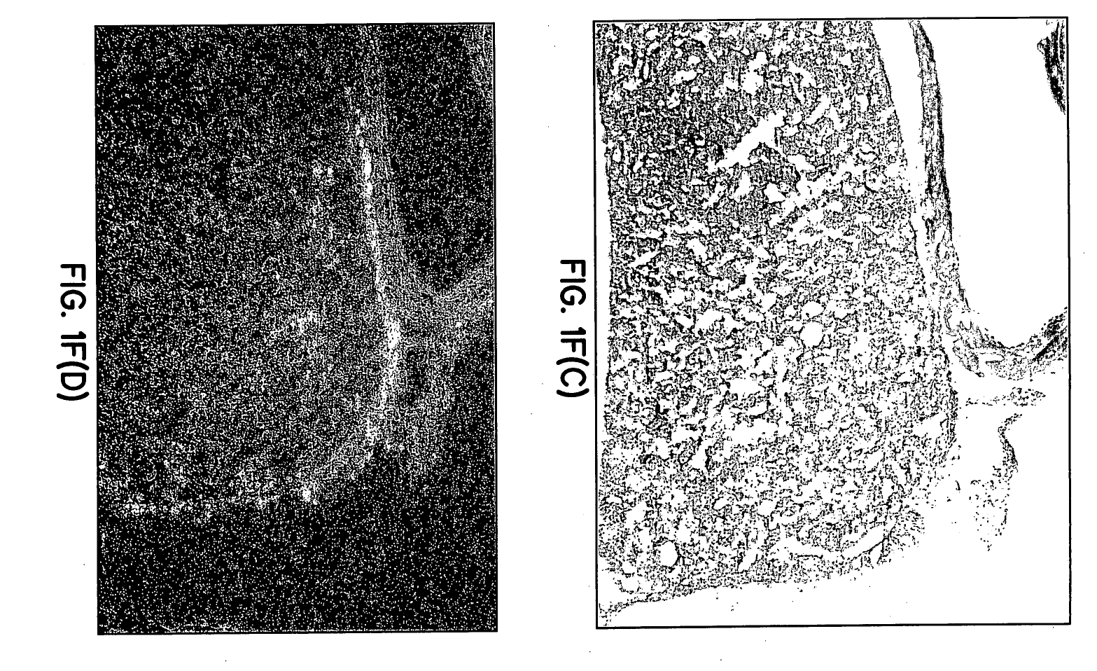

FIG. 1 shows MIG and IP-10 mRNA expression in ovalbumin (OVA) or Aspergillus fumigatus-induced asthma models. 1A shows the average difference (mean and standard error of the mean) for the hybridization signal of MIG in saline (control) and OVA challenged mice. 1B shows results for IP-10 at three (3 H) and eighteen hours (18 H) following one challenge, and eighteen hours following two challenges (2 C). 1C shows MIG and IP-10 mRNA expression in saline and OVA challenged mice. FIG. 1D shows MIG and IP-10 mRNA expression following saline or Aspergillus challenge. FIG. 1E shows MIG mRNA expression in wild-type and STAT-6 deficient mice following saline or Aspergillus challenge. The location of 18 S RNA is shown; the RNA gels were stained with ethidium bromide. Each lane represents RNA from a single mouse.

MIG mRNA was increased by >10-fold 18 h after the first allergen challenge (FIG. 1A, 18 H), and >>10-fold after the second allergen challenge (FIG. 1A, 2 C), compared to saline controls. IP-10 mRNA was increased by >10-fold 18 h after the first allergen challenge (FIG. 1B, 18 H), and >10-fold after the second allergen challenge (FIG. 1B, 2 C) compared to saline controls.

To verify that the microarray data reflected gene induction, Northern blot analysis was performed. As shown in FIG. 1C, MIG and IP-10 mRNA were strongly inducible following allergen challenge. Bands for MIG mRNA appeared at 18 h after the first challenge, and were more intense after the second challenge. Bands for IP-10 mRNA appeared 3 h after the first challenge, as well as 18 h after the first challenge and after the second challenge. Thus, the Northern blot analysis substantiated the microarray data.

To determine if the induction of MIG and IP-10 was limited to the OVA-induced model of experimental asthma, experimental asthma was induced in naive mice by repeated intranasal doses of the antigen Aspergillus fumigatus. Eighteen hours after the last of nine doses of intranasally administered Aspergillus fumigatus, lung RNA was subjected to Northern blot analysis and probed for MIG and IP-10. The results are shown in FIG. 1D.

Compared with mice challenged with nine doses of intranasal saline, mice challenged with Aspergillus fumigatus had marked expression of MIG and IP-10. Thus, the induction of MIG and IP-10 by allergen challenge was not specific to the antigen employed.

Regulation of MIG Expression

The effect of cytokines that are known to be overexpressed in the asthmatic lung, for example IL-4 and IL-13, on the induction of MIG expression, were determined. Specifically, MIG expression in transgenic mice that over-express IL-4 was determined; IL-4 overexpression did not induce MIG expression (data not shown). MIG expression in mice administered IL-13 to the lungs via the intranasal route was also determined; IL-13 administration did not induce MIG expression (data not shown). In contrast, under the same condition, IL-4 and IL-13 induced eotaxin-1 and eotaxin-2 expression.

IL-4 and IL-13 share a common receptor signaling pathway involving post-receptor events that are usually dependent on the transcription factor STAT-6. Thus, the effect of the protein STAT-6 on MIG induction was determined by inducing experimental asthma in both STAT-6 wild type mice and STAT-6 gene-deleted mice. The results are shown in FIG. 1E. Compared to STAT-6 wild-type mice, allergen-induced MIG was enhanced in STAT-6 knockout mice. These results contrasted with the induction of eotaxin-1 and eotaxin-2, which were completely dependant upon STAT-6 (data not shown).

FIG. 1F shows MIG mRNA expression in allergen-challenged lung. There was predominant peri-vascular and peri-bronchial expression of MIG RNA in OVA-challenged lung. This was seen by bright field (FIG. 1F(a)) and dark field (FIG. 1F(b)) in situ hybridization of MIG mRNA (10× magnification). FIGS. 1F(c) and 1F(d) show expression of MIG in lung lymph node from OVA-challenged lung in bright field and dark field in situ hybridization, respectively (40× magnification).

Murine MIG is an Inhibitor of Eosinophils in vitro

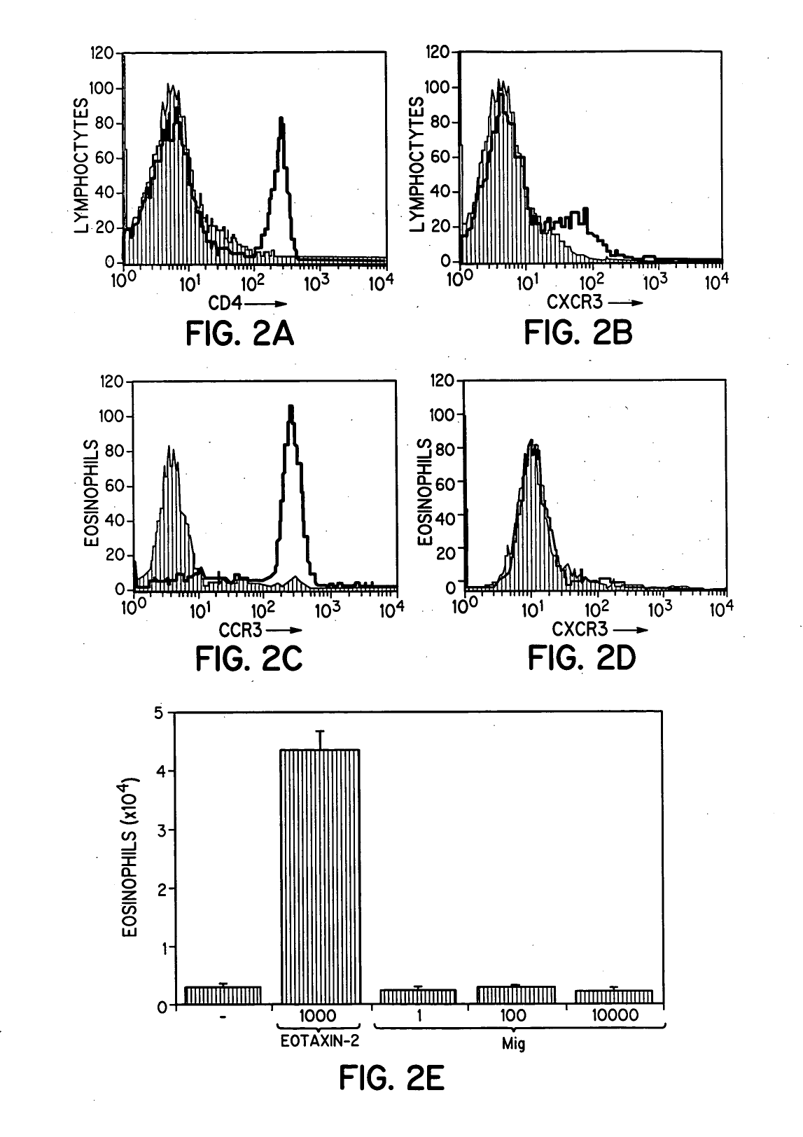

Human eosinophils have been reported to express CXCR3, which is the receptor for MIG. To determine if allergen-induced expression of MIG could be responsible, at least in part, for recruitment of eosinophils to the lung, it was first determined whether murine eosinophils expressed CXCR3. The results showing failure of murine eosinophils, which lack CXCR3 on their surface, to migrate toward MIG are shown in FIG. 2. FIG. 2A shows lymphocytes from IL-5 transgenic mice which express CXCR3; eosinophils have no detectable CXCR3 on their surface. The filled histogram is the isotope-matched control, and the solid line is CCR3, CXCR3, or CD4. FIG. 2B shows a representative result (n=3) of transmigration of spleen-derived eosinophils in response to doses of MIG as indicated. Cells (1.5×10⁶) were allowed to transmigrate in response to MIG and eotaxin-2. Cells were counted in the lower chamber 1.5 hours later. Data represent mean and standard deviation of eosinophils that migrated through a layer of respiratory epithelial cells.

The histograms in FIG. 2A show flow cytometry data from eosinophils and lymphocytes. CD4+ lymphocytes expressed the CXCR3 receptor on the cell surface. In contrast, eosinophils, identified by their characteristic light scatter and expression of the eotaxin receptor CCR3, had no detectable expression of the MIG receptor CXCR3. In contrast to the strong expression of CCR3 in eosinophils, there was no reproducible staining for CXCR3 on eosinophils (n=3 experiments) using a range of antibody doses (0.5 μg/ml to 30 μg/ml). As a control, CXCR3 was identified in CD4+ T cells.

It was next determined if MIG could induce eosinophil migration in vitro. FIG. 2B shows a representative transmigration assay for lung eosinophils. Consistent with the absence of CXCR3 expression, murine eosinophils did not respond by transmigration when subjected to a range of doses of MIG (1 pg/ml to 10,000 pg/ml). As a positive control, eosinophils strongly responded to 1000 pg/ml of eotaxin-2. These data suggest that MIG was not a stimulatory chemokine for murine eosinophils.

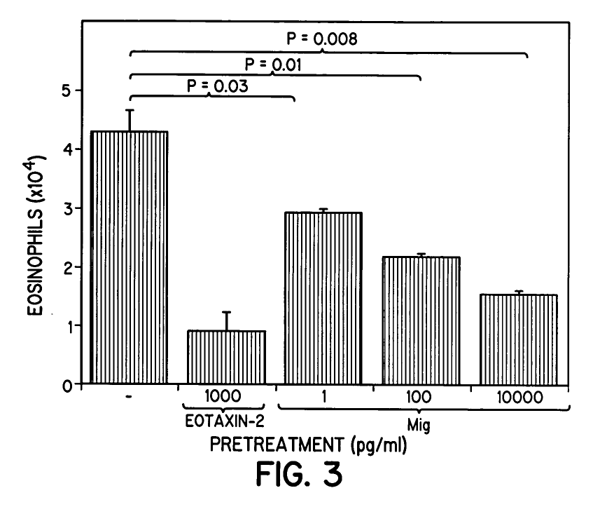

MIG was, however, a functional inhibitor for CCR3 ligand-induced eosinophil chemoattraction in vitro. Eosinophils were pretreated with MIG, and their subsequent chemotactic response to the potent CCR3 ligand, eotaxin-2, was evaluated. The results are shown in FIG. 3.

FIG. 3 shows data for eosinophil transmigration in response to eotaxin-2; MIG inhibited eosinophil migration toward eotaxin-2 in vitro. Cells were allowed to transmigrate following pretreatment with buffer, MIG, or eotaxin-2. Data (mean and standard deviation) are from a representative experiment (n=3) of eosinophils that migrated toward 1 ng/ml eotaxin-2.

Pretreatment of the eosinophils with MIG inhibited eosinophil transmigration in response to eotaxin-2. This effect was seen in a dose-dependent manner, with inhibition of activity noted between 1 pg/ml to 10,000 pg/ml (0.8 pM to 820 pM); p=0.03 at 1 pg/ml, p=0.01 at 100 pg/ml, and p=0.008 at 10,000 pg/ml MIG. As a positive control, pretreatment of eosinophils with eotaxin-2 at a dose of 1 ng/ml (0.1 nM) also inhibited eosinophil transmigration. As a negative control, pretreatment of eosinophils with MCP-1 (JE, CCL2) at a dose of 10 ng/ml (0.7 nM) did not inhibit eosinophil transmigration (data not shown). MIG was not toxic to eosinophils, as determined by exclusion of a viability dye (Trypan blue), and by the ability of IL-5 to promote eosinophil survival even in the presence of MIG (data not shown).

Effect of MIG on CCR3 Internalization

CCR3 ligands induce receptor internalization following receptor engagement with agonists. MlG-induced CCR3 internalization could account for the ability of MIG to inhibit the transmigration of eosinophils that are induced by eotaxin.

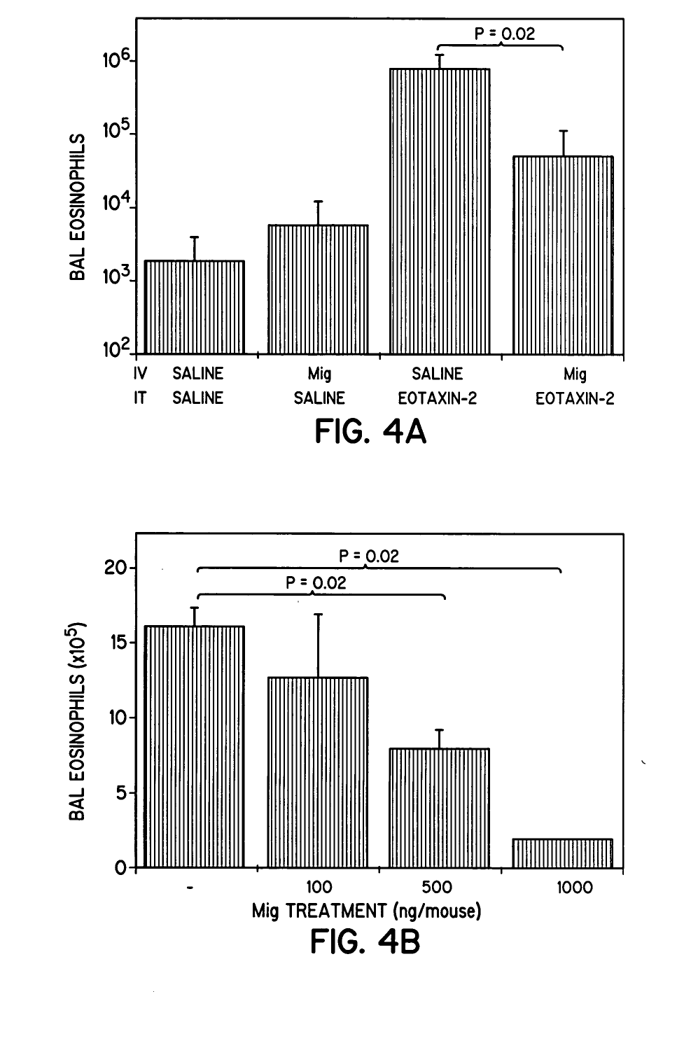

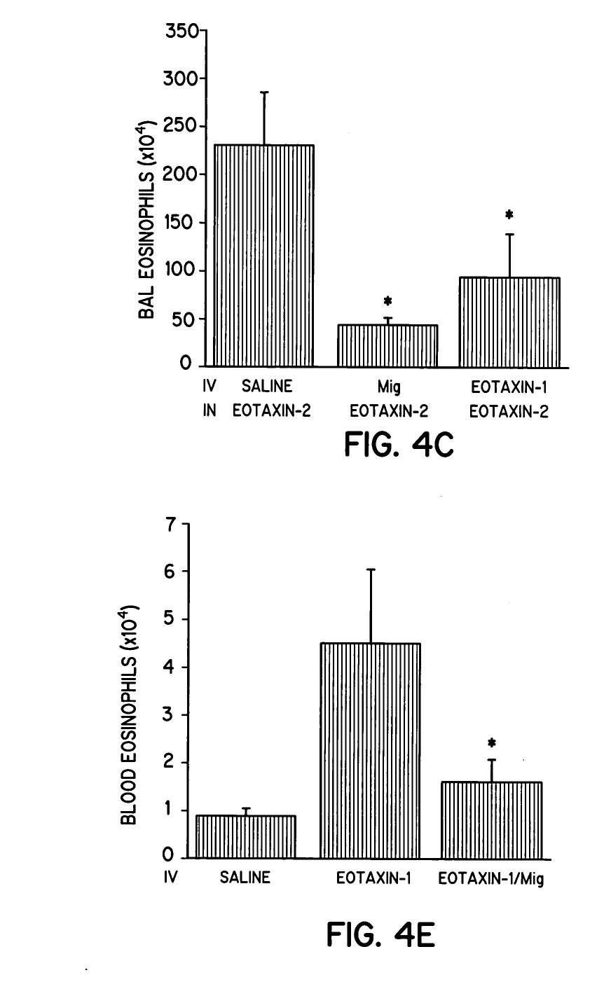

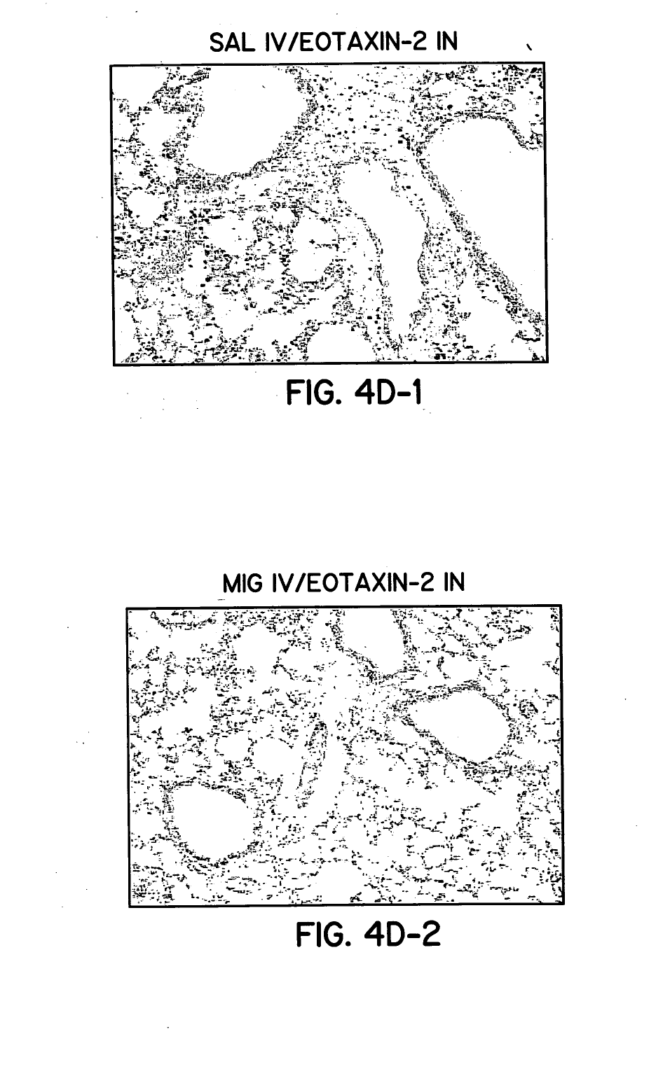

FIG. 4 shows dose-dependent MIG inhibition of chemokine-induced eosinophil recruitment to the lung. FIG. 4A shows the mean and standard deviation of eosinophils that migrated into the airway towards eotaxin-2. IL-5 transgenic mice were treated intravenously with saline or 1 pg MIG thirty minutes prior to intratracheal challenge with 3 μg eotaxin-2 or saline. Data represent three independent experiments, with two to six mice in each group. FIG. 4B shows mice treated with saline or MIG at the doses indicated prior to challenge with eotaxin-2. Data (mean and standard deviation) show airway eosinophils from a representative experiment (n=2), with four mice in each group per experiment.

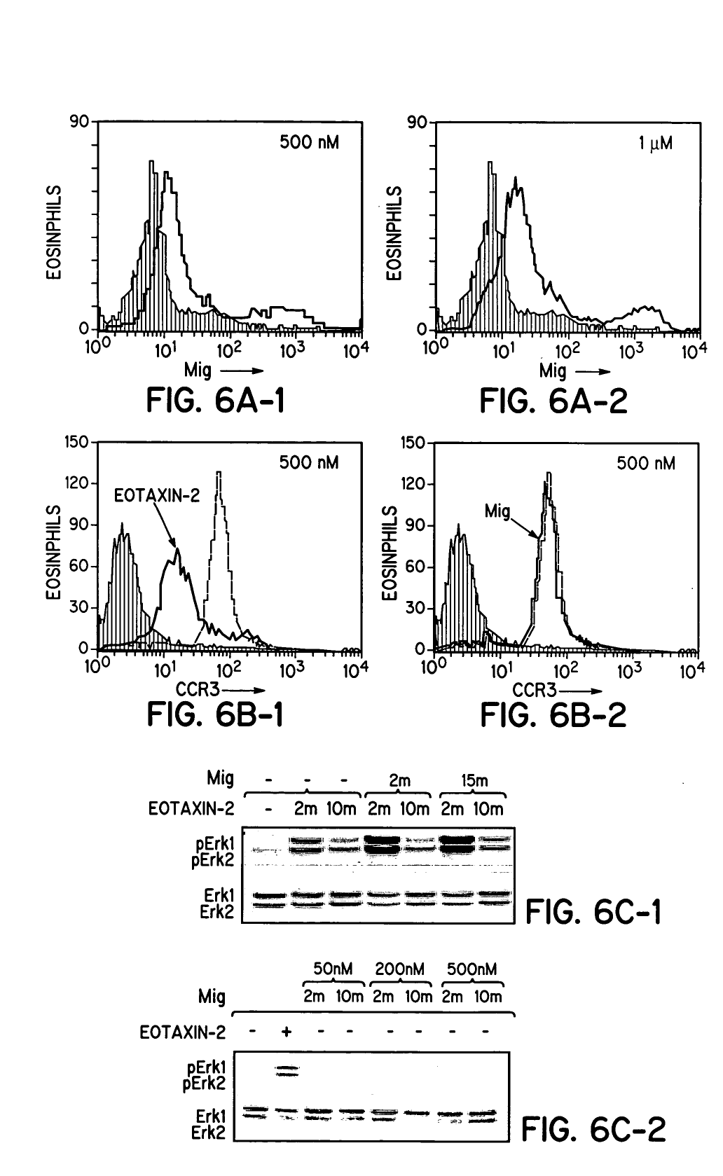

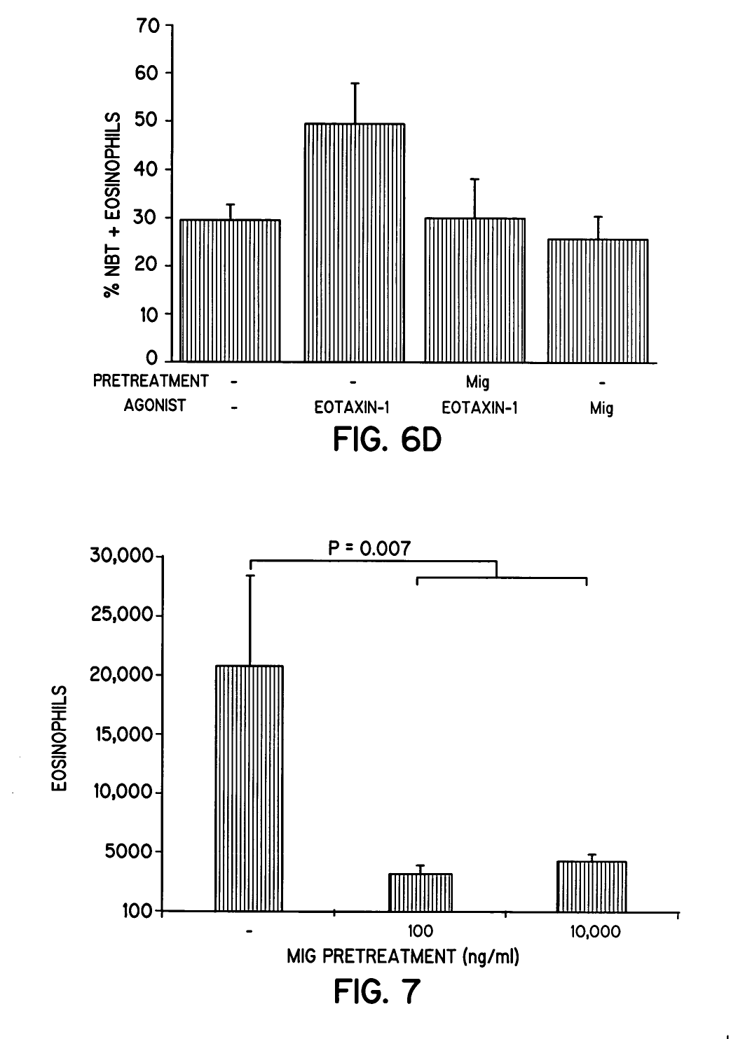

As shown in FIG. 6B, pretreatment of eosinophils with MIG at doses of 1 ng/ml, 100 ng/ml, or 1000 ng/ml (82 pM to 82 nM) did not significantly affect the level of CCR3. As a control, pretreatment of eosinophils with 100 ng/ml (9.7 nM) eotaxin-2 induced marked internalization of CCR3 expression. The effect of eotaxin-2 was not seen when the pre-incubation was conducted at 4° C., verifying that the assay was detecting receptor internalization rather than epitope blockade.

MIG Inhibits Eosinophil Recruitment to the Lung Induced by Eotaxin and IL-13

The effect of MIG to serve as an eosinophil inhibitor in vivo was determined. The ability of MIG to inhibit eosinophil recruitment into the lung, induced by either eotaxin-2 or IL-13, was evaluated. Pretreatment of mice with 1 μg MIG administered intravenously thirty minutes prior to eotaxin-2 administered intratracheally resulted in >90% inhibition in eosinophil trafficking to the lung (FIG. 4A). MIG demonstrated dose-dependent inhibition between 0.1 μg MIG to 1.0 μg MIG (FIG. 4B).

Eotaxin-2, administered intratracheally to IL-5 transgenic mice, induced marked recruitment of eosinophils into the lung. For example, and with reference to FIG. 4A, eosinophil levels in BALF three hours after eotaxin-2 treatment increased from 1.9±2.3×10³ (n=2 mice) to 7.2±5.4×10⁵ (n=6 mice). However, intravenous injection of mice with MIG (1 μg) thirty minutes prior to intratracheal eotaxin-2 administration reduced recruitment of eosinophils, compared to intravenous injection of saline (p<0.02).

Intravenous administration of MIG thirty minutes prior to intranasal administration of eotaxin-2 inhibited eosinophil recruitment into the lung in a dose-dependent manner. With reference to FIG. 4B, intravenous administration of MIG to IL-5 transgenic mice at a dose of 100 ng reduced eosinophil recruitment into BALF by 21%. Intravenous administration of MIG at a dose of 500 ng reduced eosinophil recruitment into BALF by 51% (p=0.02). Intravenous administration of MIG at a dose of 1000 ng reduced eosinophil recruitment into BALF by 88% (p=0.01). To verify that MIG was specifically responsible for the inhibitory activity, mice were treated with a different chemokine, murine MCP-1 (also known as JE) (1 μg), prior to intranasal administration of eotaxin-2. The chemokine JE had no effect on eotaxin-induced eosinophil recruitment in the lung (data not shown). For comparison, mice were intravenously administered 1 μg of eotaxin-1 prior to intranasal eotaxin-2 administration, and eosinophil migration into lung in response was determined. As shown in FIG. 4C, MIG and eotaxin-1 had comparable inhibitory activity. Data represent mean ± standard of deviation of lung or airway eosinophils in a representative experiment (n=2) with four mice in each group per experiment. (*p≦0.04).

Eosinophil levels in lung tissue were assessed by histological examination and by anti-MBP staining. Eosinophil migration was inhibited after intravenous MIG treatment prior to eotaxin-2 intranasal delivery. The results are shown in FIG. 4D with eosinophils detected by anti-MBP immunohistochemistry in lung following intravenous treatment of saline or MIG prior to intranasal administration of eotaxin-2. MIG (1 μg) administered intranasally prior to eotaxin-2 administration did not significantly inhibit eosinophil recruitment (data not shown). Thus, MIG activity appeared to depend on systemic, rather than local, administration. In contrast to the inhibitory effect of MIG on eosinophil recruitment into either BALF or lung tissue, eosinophil levels in the blood were not affected by MIG at any of the doses administered.

The ability of MIG to inhibit eosinophil chemokine responses in vivo was not limited to eotaxin-2; MIG also inhibited the effects of eotaxin-1. Pretreatment with 1 μg MIG reduced eotaxin-1 induced BALF eosinophilia from 2.6±0.42×10⁶ to 4.0±1.6×10⁵ cells (n=3 mice/group). MIG also inhibited eotaxin-induced eosinophil mobilization to the blood. After intravenous administration of 1 μg eotaxin-1, eotaxin-1 induced a rapid increase in circulating eosinophil levels. The results are shown in FIG. 4E. When 1 μg MIG was administered in combination with eotaxin-1, eotaxin-induced eosinophil mobilization was significantly reduced (*p<0.0001) (3 experiments with 12 mice in each group).

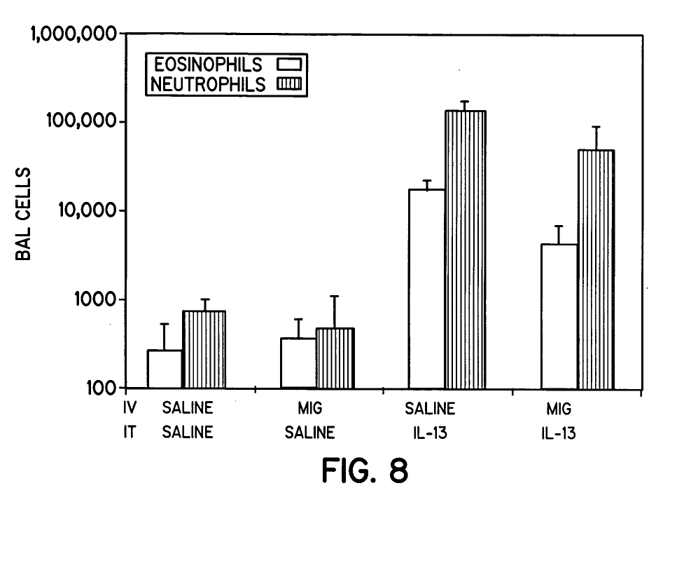

MIG inhibited IL-13-induced granulocyte trafficking to the lung in vivo. IL-13 treated mice were treated intravenously with saline or MIG thirty minutes prior to a second dose of IL-13. The results are shown in FIG. 8; data represent the mean and standard deviation of eosinophils and neutrophils in BALF following treatment, and show a representative experiment (n=2).

IL-13, administered intratracheally to naive Balb/c mice, also induced marked recruitment of eosinophils into the lung. IL-13 was administered at a dose of 4 μg. After two days, the mice were administered an intravenous injection of MIG (1 μg), followed by a second dose of IL-13 (10 μg), again administered intratracheally. After three days, the cell content of the BALF was examined.

With reference to FIG. 8, BALF from IL-13 dosed mice pretreated with MIG (MIG IV/IL-13 IT) had decreased eosinophils (4.2±2.7×10³ (n=4 mice) compared to mice pretreated with saline (control, Sal IV/IL-13 IT) (17.8±5.0×10³ (n=4 mice) (p=0.003). BALF from mice treated with IL-13 following treatment with MIG also had decreased neutrophils (5.1±3.7×10⁴ (n=4 mice) compared to mice pretreated with saline (control, Sal IV/IL-13 IT) (13.2±3.6×10⁴ (n=4 mice) (p=0.02). These data indicate that intravenous MIG inhibited eosinophil recruitment into the lung in response to diverse stimuli. The finding that neutrophil levels in the lung were also inhibited by MIG suggests its generalized ability to block leukocyte trafficking, and more particularly granulocyte trafficking, to the lung. The ability of MIG to block the action of IL-13 is beneficial from a therapeutic vantage, because IL-13 is considered to be a central and critical cytokine in the pathogenesis of asthma.

MIG Inhibits OVA-Induced Eosinophil Recruitment to the Lung

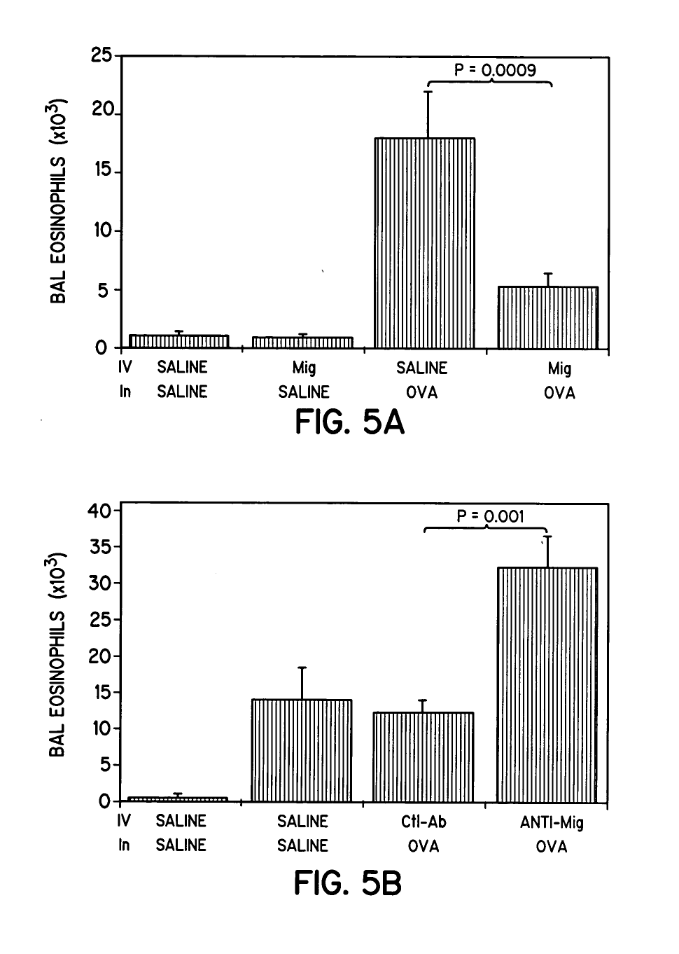

To determine if pharmacological administration of MIG down-regulated eosinophil recruitment to the lung in the OVA-induced experimental asthma model, mice sensitized with OVA were subjected to one challenge with intranasal OVA or saline. The ability of MIG, administered intravenously 30 minutes prior to allergen challenge, to inhibit leukocyte recruitment into the lung was determined. FIG. 5 shows that MIG inhibited allergen-induced eosinophil recruitment to the lung and functioned as an eosinophil inhibitor in vivo. FIG. 5A shows a representative experiment (n=2), with four mice in each group per experiment, of OVA-challenged mice treated with intravenous saline or MIG (1 μg) thirty minutes prior to intranasal OVA challenge. FIG. 5B shows MIG neutralization increased antigen-induced eosinophil recruitment to the lung. OVA-challenged mice were treated with an intraperitoneal injection of 500 μg anti-MIG antibody or isotope-matched control antibody (Ctl-Ab). The results are from a representative experiment (n=2) with two to four mice in each group per experiment. Data from FIGS. 5A and 5B represent the mean and standard deviation of airway eosinophils.

Control mice (saline injection) challenged with OVA demonstrated an increased total leukocyte count in BALF. Eosinophils in BALF increased from 9.1±4.0×10² (IV Saline, IN Saline) to 1.7±0.4×10⁴ (IV Saline, IN OVA)(p=0.08). Total leukocytes in BALF increased from 5.3±0.7×10⁴(n=3) to 10.2±2.1×10⁴ (n=4). Neutrophils increased in BALF increased from 8.1±4.4×10³ to 4.7±1.4×10⁴ (p=0.005).

Mice administered MIG thirty minutes prior to challenge with OVA had decreased eosinophils in BALF. In comparison to mice receiving saline prior to OVA challenge (IV Saline, IN OVA), mice receiving MIG prior to OVA challenge (IV MIG, IN OVA) demonstrated a 70% reduction (p=0.0009) in eosinophils in BALF. This reduction was specific to eosinophils; mice receiving MIG prior to OVA challenge had no reduction in either BALF neutrophils or lymphocytes (data not shown). This reduction was also specific to MIG; mice receiving the cytokine JE (1 μg) prior to OVA challenge showed no change compared to mice receiving saline prior to OVA challenge (data not shown).

The ability of endogenously expressed MIG, in contrast to pharmaceutically administered MIG to inhibit eosinophil migration in vivo was assessed. It was expected that OVA-challenged mice with decreased or absent MIG would demonstrate increased eosinophil recruitment into the BALF. Mice sensitized with OVA were administered anti-murine MIG (500 μg) twenty-four hours prior to one intranasal challenge with OVA. Eosinophil recruitment into the BALF was then evaluated. The results are shown in FIG. 5B.

IgG control treated mice, challenged with OVA, increased eosinophil recruitment into the airway. Eosinophils in BALF in control mice were 5.0±4.5×10² (IP Saline, IN Saline), while eosinophils in BALF in mice receiving a control antibody (IP CTL-Ab, IN OVA) were 1.2±0.2×10⁴. Treatment of mice with anti-MIG antibody increased eosinophils in BALF greater than two-fold over isotope control treated mice. Eosinophils in BALF in isotope control treated mice were 1.2±0.2×10⁴, while eosinophils in BALF in anti-MIG treated mice were 3.3±0.4×10⁴, following OVA challenge.

Direct Binding of MIG on Murine Eosinophils

The nature of the effect of MIG on eosinophils was determined by evaluating whether MIG bound to eosinophils in vitro. Eosinophil preparations were prepared from the spleen of IL-5 transgenic mice; those mice have large numbers of eosinophils in the spleen and serve as a convenient source of murine eosinophils. Splenocytes were exposed to MIG at one of four doses (100 nM to 1 μM). The binding of MIG to splenocyte eosinophils was evaluated by flow cytometry using anti-MIG antiserum. MIG bound to the surface of eosinophils and attenuated eotaxin-2 signal transduction, as shown in FIG. 6A. MIG bound in a dose-dependent manner to the surface of murine eosinophils from IL-5 transgenic mice. In comparison, the filled histogram represents no binding when no chemokine is present. FIG. 6B shows that MIG did not induce CCR3 internalization. Analysis of surface CCR3 on eosinophils following incubation with buffer (solid line), eotaxin-2 (dotted line), or MIG (dashed line). The filled histogram shows results from isotope matched controls. FIG. 6C shows enhanced eotaxin-2 induced phosphorylation of p44/42 (Erk 1 and Erk 2) in eosinophils following MIG pretreatment. Cells were incubated with buffer, MIG, and/or eotaxin-2 at the indicated time and dose. Phosphorylation of p44/42 was determined by Western blot analysis. The results are from a representative experiment (n=2).

Increased doses of MIG resulted in increased binding of MIG to the surface of eosinophils, compared to eosinophils that were not exposed to MIG or another cytokine. Exposure of eosinophils to 500 nM and 1 μM MIG showed a dose-dependent increase in MIG binding. As a negative control, eosinophils were exposed to the cytokine JE, a ligand of CCR2 which is not normally expressed by murine eosinophils. Binding of MIG to eosinophils was greater than binding of JE to eosinophils (data not shown), even in the absence of detectable expression of CXCR3 on the surface of murine eosinophils.

To determine if CCR3 was the MIG receptor in eosinophils, the ability of MIG to compete for the binding of biotinylated eotaxin-1 to eosinophils was determined. While unlabeled eotaxin-1 or eotaxin-2 was able to compete for biotinylated eotaxin-1 binding to eosinophils, unlabeled MIG at doses up to 10 μm did not inhibit the binding CCR3 ligands (data not shown). Because MIG interaction with CCR3 should result in decreased binding of MIG to eosinophils with internalization of CCR3, the effect of MIG binding was determined with eosinophils that had been pretreated with eotaxin-2 under conditions that promoted marked CCR3 internalization. Under these conditions, MIG binding was not inhibited (data not shown). Therefore, in contrast to the proposal set forth by Loetscher et al. (J. Biol. Chem. 276:2986 (2001), the ability of MIG to inhibit eosinophil responses was not simply related to competitive antagonism of CCR3.

Activation of receptors by chemokines leads to a cascade of intracellular signaling and multiple phosphorylation events. Mitogen activated protein (MAP) kinases are phosphorylated and activated after exposing human eosinophils to CCR3 ligands. The effect of MIG on CCR3-ligand induced signal transduction was therefore determined. Specifically, the phosphorylation activity of two MAP kinases (p44 MAP kinase phosphorylating Erk1, and p42 MAP kinase phosphorylating Erk2) was assayed in eosinophils exposed to eotaxin-2 in the presence and absence of MIG pretreatment. The results are shown as Western blots in FIG. 6C.

In control eosinophils (exposed to eotaxin-2 without MIG pretreatment), phosphorylation by both p44 and p42 was maximal at two minutes, and then was down-regulated at ten minutes after eotaxin-2 exposure. No phosphorylation by either p44 or p42 was detected with only MIG exposure (no eotaxin-2) at any MIG dose (50 nM to 100 nM). FIG. 6C shows the effect of pretreatment with MIG at 50 nM. Eosinophils pretreated with MIG, followed by eotaxin-2 exposure, demonstrated increased Erk1 and Erk2 phosphorylation compared with eosinophils exposed to eotaxin-2 above.

MIG Inhibits Functional Response in Eosinophils

Eosinophils were pretreated with MIG and then treated with eotaxin-1 (10 nM). Eotaxin activation of eosinophils increased nitrobluetetrazolium positive (NBT⁺) cells, indicating superoxide anion and related reactive oxygen species. As shown in FIG. 6D, MIG (100 nM) pretreatment inhibited eotaxin-induced NBT⁺ eosinophils by 94%. Results represent percentage of positive cells, with error bars showing mean ± standard deviation, n=3.

MIG Inhibits Eosinophil Responses to Diverse Chemoattractants.

The inhibition of MIG on allergen-induced eosinophilia in the lung was surprising in view of the recent finding that CCR3 deficient mice exhibited about a 50% reduction in BALF eosinophilia following sensitization and challenge with OVA. Therefore, in addition to inhibiting CCR3-mediated pathways in eosinophils, MIG could also inhibit chemoattractants that signal through additional pathways. This was consistent with results that MIG also blocked IL-13-induced eosinophil lung recruitment, because IL-13 induces multiple eosinophil chemoattractants.

The ability of MIG to alter chemotaxis of eosinophils toward a non-CCR3 ligand was evaluated. Eosinophils were pretreated with MIG at a dose of either 100 ng/ml or 10000 ng/ml. Their subsequent chemotactic response to platelet activating factor (PAF) (1 μm) was evaluated. FIG. 7 shows that MIG inhibited migration of eosinophils toward PAF in vivo. Cells were allowed to transmigrate toward PAF following pretreatment with buffer or MIG. The data (mean and standard deviation) are from a representative experiment (n=2) and show eosinophils that migrated toward 1 μm PAF.

As shown in FIG. 7, MIG pre-treatment inhibited eosinophil migration in response to PAF (p=0.007). Thus, MIG induced inhibition was not limited to CCR-3 ligands, and altered eosinophil responses to diverse chemoattractants. These data indicated that MIG induced functional non-responsiveness of eosinophils. While not being bound by a specific theory, such non-responsiveness may be due to heterologous receptor desensitization.

The composition may be administered to a mammal, such as a human, either prophylactically or in response to a specific condition or disease. For example, the composition may be administered to a patient with asthmatic symptoms and/or allergic symptoms. The composition may be administered non-systemically such as by inhalation, aerosol, drops, etc.; systemically by an enteral or parenteral route, including but not limited to intravenous injection, subcutaneous injection, intramuscular injection, intraperitoneal injection, oral administration in a solid or liquid form (tablets (chewable, dissolvable, etc.), capsules (hard or soft gel), pills, syrups, elixirs, emulsions, suspensions, etc.). As known to one skilled in the art, the composition may contain excipients, including but not limited to pharmaceutically acceptable buffers, emulsifiers, surfactants, electrolytes such as sodium chloride; enteral formulations may contain thixotropic agents, flavoring agents, and other ingredients for enhancing organoleptic qualities.

The dose of MIG administered in the composition to a mammal is in the range between about 10 μg/kg to about 10 mg/kg. The dose of IP-10 administered to a mammal is in the range between about 10 μg/kg to about 10 mg/kg. In one embodiment, a dose of about 30 μg/kg of MIG or IP-10 is administered. Dosing may be dependent upon the route of administration. As examples, an intravenous administration may be continuous or non-continuous; injections may be administered at convenient intervals such as daily, weekly, monthly, etc.; enteral formulations may be administered once a day, twice a day, etc. Instructions for administration may be according to a defined dosing schedule, or an “as needed” basis.

Other variations or embodiments of the invention will also be apparent to one of ordinary skill in the art from the above figures and descriptions. For example, linear peptides with homology to MIG and/or IP-10, exhibiting a similar functional activity as MIG and/or IP-10 (analogues of MIG and/or IP-10), may also be administered. Thus, the forgoing embodiments are not to be construed as limiting the scope of this invention.

What is claimed is:

1. A method of inhibiting at least one of eosinophil recruitment or eosinophil function comprising administering a pharmaceutical composition comprising an isolated cytokine with eosinophil recruitment- or function-inhibitory activity in a pharmaceutically effective amount to inhibit eosinophil recruitment or eosinophil function selected from the group consisting of receptor expression, receptor internalization, signal transduction, transmigration, desensitization, degranulation, mediator release, oxidase activity, and combinations thereof.

2. The method of claim 1 wherein the isolated cytokine is selected from the group consisting of monokine induced by interferon γ (MIG), IFN-γ-inducible protein of 10 kDa (IP-10), and combinations thereof.

3. The method of claim 1 wherein the isolated cytokine is a peptide derived from MIG or IP-10.

4. The method of claim 1 wherein the isolated cytokine is a protein homologous to MIG or IP-10.

5. The method of claim 1 wherein the composition is administered to an individual with eosinophilia.

6. The method of claim 1 wherein a signal transduction kinase function is perturbed.

7. The method of claim 1 wherein Erk1 or Erk2 is perturbed.

8. The method of claim 1 wherein transmigration is altered in at least one of lung, trachea, airway, bronchoalveolar lavage fluid, heart, or skin.

9. A method of reducing allergen-induced eosinophilia in a mammal comprising administering to a mammal exposed to an allergen a pharmaceutical composition comprising an isolated monokine induced by interferon γ (MIG) and/or an IFN-γ-inducible protein of 10 kDa (IP-10) in a pharmaceutically effective amount to reduce allergen-induced eosinophilia.

10. The method of claim 9 wherein eosinophilia is reduced in an airway, lung, trachea, bronchoalveolar lavage fluid, or blood.

11. The method of claim 9 wherein eosinophilia is reduced in a body part affected by an allergy.

12. The method of claim 11 wherein the body part is selected from the group consisting of skin, eye, nose, gut, and combinations thereof.

13. The method of claim 9 wherein the mammal is a human.

14. A treatment method comprising administering to an individual a pharmaceutical composition comprising an isolated eosinophil-inhibitory cytokine in an amount sufficient to inhibit an eosinophil response to a chemoaftractant.

15. The method of claim 14 wherein the cytokine is selected from the group consisting of monokine induced by interferon γ (MIG), an IFN-γ-inducible protein of 10 kDa (IP-10), or combinations thereof.

16. The method of claim 14 wherein the chemoattractant is selected from the group consisting of eotaxin-1, eotaxin-2, eotaxin-3, MCP-2, MCP-3, MCP-4, MCP-5, RANTES, MIP-1a, and combinations thereof.

17. The method of claim 14 wherein the cytokine is administered at a dose in the range of about 10 μg/kg to about 10 mg/kg.

18. The method of claim 14 wherein the cytokine is administered at a dose of about 30 μg/kg.

19. The method of claim 14 wherein the cytokine is administered systemically.

20. The method of claim 14 wherein the cytokine is administered by a route selected from the group consisting of intravenously, intranasally, intratracheally, subcutaneously, intramuscularly, orally, intraperitonally, and combinations thereof.

21. A palliative method comprising administering a pharmaceutical composition comprising an isolated eosinophil-inhibitory cytokine in an amount to alleviate inflammation in at least one of an airway or tissue of a patient exposed to an allergen or having an eosinophilic syndrome.

22. The method of claim 21 wherein the eosinophil-inhibitory cytokine is monokine induced by interferon γ (MIG) and/or an IFN-γ-inducible protein of 10 kDa (IP-10).

23. The method of claim 21 wherein the allergen results in a condition selected from the group consisting of allergic rhinitis, asthma, eczema, and combinations thereof.

24. A method of inhibiting pulmonary eosinophil recruitment comprising administering to a mammal a pharmaceutical composition comprising an isolated monokine induced by interferon γ (MIG) and/or an IFN-γ-inducible protein of 10 kDa (IP-10) in a pharmaceutically effective amount to inhibit pulmonary eosinophil recruitment.

25. The method of claim 24 administered prophylactically to an asthmatic individual.

26. The method of claim 24 administered therapeutically to an asthmatic individual.

27. A treatment method comprising providing to a patient an amount and formulation of a pharmaceutical composition containing at least one cytokine capable of negatively regulating an inflammatory cell within a lung of the patient.

28. The method of claim 27 wherein the patient is asthmatic, allergic, or has a hypereosinophilic disease.

29. A treatment method comprising administering to an individual with eosinophilia a cytokine selected from the group consisting of monokine induced by interferon γ (MIG), an IFN-γ-inducible protein of 10 kDa (IP-10), or combinations thereof in a pharmaceutically acceptable formulation at a cytokine dose of up to about 10 mg/kg.

30. The method of claim 29 wherein the cytokine alters an eosinophil function selected from the group consisting of migration, tissue recruitment, receptor binding, signal transduction, degranulation, mediator release, and combinations thereof.

31. The method of claim 29 wherein recruitment is responsive to an allergen and/or chemokine.

32. The method of claim 29 wherein MIG or IP-10 is administered to an asthmatic patient.

33. The method of claim 29 wherein MIG or IP-10 is administered to an allergic patient.

34. A method for alleviating asthma in a patient comprising administering to an asthmatic patient monokine induced by interferon γ (MIG) in a pharmaceutical composition thereby inhibiting an IL-13-associated asthmatic response in the patient.

35. A pharmaceutical composition comprising an isolated monokine induced by interferon γ (MIG) and/or an IFN-γ-inducible protein of 10 kDa (IP-10) in a pharmaceutically acceptable formulation and an amount sufficient to alter eosinophil activity in the presence of an allergen.

36. The composition of claim 35 wherein the amount is a dose in the range of about 10 μg/kg to about 10 mg/kg.

37. A pharmaceutical composition comprising a cytokine which inhibits at least one eosinophil function in response to an eosinophil-induced stimulus.

38. The composition of claim 37 wherein the cytokine is selected from the group consisting of monokine induced by interferon γ (MIG), an IFN-γ-inducible protein of 10 kDa (IP-10), or combinations thereof.

39. The composition of claim 37 wherein the stimulus is selected from the group consisting of an allergen, a chemokine, a cytokine, and combinations thereof.

40. The composition of claim 37 wherein the stimulus is selected from the group consisting of eotaxin-1, eotaxin-2, eotaxin-3, IL-13, platelet activating factor, and combinations thereof.

41. The composition of claim 37 wherein the stimulus is an allergic reaction, an infection, idiopathic eosinophilia, and combinations thereof.

42. A pharmaceutical composition comprising an isolated Th1-associated chemokine in a pharmaceutically acceptable formulation and in an amount sufficient to inhibit eosinophil activity in the presence of an allergen.

43. A pharmaceutical composition comprising a cytokine selected from the group consisting of recombinant monokine produced by interferon γ (MIG), a recombinant IFN-γ-inducible protein of 10 kDa (IP-10), and combinations thereof in a pharmaceutically acceptable formulation and dose sufficient to inhibit an eosinophil function.

44. A method of reducing in vivo eosinophil chemoattraction comprising administering to a patient a pharmaceutically acceptable formulation of a cytokine substantially lacking eosinophil chemoattraction activity and negatively affecting at least one of eosinophil chemoattraction or eosinophil activation activity.

45. The method of claim 44 wherein the cytokine substantially lacking eosinophil chemoattraction is a Th1 cytokine.

46. The method of claim 44 wherein the cytokine substantially lacking eosinophil chemoattraction is at least one of MIG and IP-10.

47. The method of claim 44 wherein a Th2 cytokine is negatively affected.

48. The method of claim 44 wherein at least one of eotaxin-1, eotaxin-2, eotaxin-3, MCP-1, MCP-2, IL-4, IL-13, and platelet activating factor (PAF) is negatively affected.

49. A method of selectively treating an eosinophil associated disease in a patient comprising administering to a patient a Th1 associated chemokine thereby inhibiting eosinophil recruitment or eosinophil function.

50. The method of claim 49 wherein the Th1 associated chemokine is MIG or IP-10.
